(19) United States
(12) Patent Application Publication
(10) Pub. No.: US 2024/0345052 A1
(43) Pub. Date: Oct. 17, 2024

(54) REAL-TIME WEARABLE SENSOR FOR MONITORING PLANT DISEASE AND STRESS

(71) Applicant: North Carolina State University, Raleigh, NC (US)

(72) Inventors: Qingshan Wei, Cary, NC (US); Yong Zhu, Apex, NC (US); Giwon Lee, Raleigh, NC (US)

(21) Appl. No.: 18/631,265

(22) Filed: Apr. 10, 2024

Related U.S. Application Data
(60) Provisional application No. 63/458,548, filed on Apr. 11, 2023.

(51) Int. Cl. G01N 33/00

(52) U.S. Cl. CPC G01N 33/0098; B82Y 15/00

(57) ABSTRACT

The present invention provides wearable plant sensors for continuous monitoring of plant physiology by tracking both biochemical and biophysical signals of the plant and its microenvironment. Sensors for detecting volatile organic compounds (VOC), temperature, and humidity are integrated into a single platform. The abaxial leaf attachment position is selected based on the stomata density to improve the sensor signal strength. This versatile platform enables various stress monitoring applications, ranging from tracking plant water loss to early detection of plant pathogens. A machine learning model was also developed to analyze multichannel sensor data for quantitative detection of tomato spotted wilt virus (TSWV) as early as four days after inoculation.

### CROSS-REFERENCE

This application claims the benefit of Provisional Application No. 63/458,548, filed on Apr. 11, 2023, which is incorporated herein by reference in its entirety.

This invention was made with government support under grant number 2019-67030-29311 awarded by the National Institute of Food and Agriculture, grant numbers CMMI2134664 and CMMI1728370 awarded by the National Science Foundation, and under grant numbers AP20PPQS&T00C062 and AP21PPQS&T00C020 awarded by the USDA Animal and Plant Health Inspection Service. The government has certain rights in the invention.

### TECHNICAL FIELD

This invention relates to wearable, real-time, sensing devices that can be used to monitor both abiotic and biotic stressors on plants.

### BACKGROUND

Imaging or spectroscopic sensors are among the few possible solutions that are capable of real-time and noninvasive monitoring of plants. However, the imaging techniques are indirect by measuring the optical signature of the plant. Such measurement possesses some obvious limitations, such as poor sensitivity/selectivity and complicated processing of raw images or spectral data. Other approaches, including remoting sensing and electrophysiological sensing, have also been proposed for continuous monitoring. However, remoting sensing in general lacks spatial solution and is not specific for particular diseases, while electrophysiological sensors have only been demonstrated for tracking water stress or the nycthemeral rhythm of the plant. Therefore, novel sensor technologies that can track the status of plant health in real-time and dissect various biotic/abiotic stresses are needed to detect pathogens early, prevent disease outbreak, and improve plant growth and yield.

The physiological status of a plant is related to multiple factors. Each plant grows via a set of biological processes such as photosynthesis, transpiration, respiration, and gas exchange through the regulation of leaf epidermal pores called stomata. To accurately monitor plant health status, many of those biological processes and associated environmental conditions need to be investigated simultaneously.

There is a need for sensor technology that can be used in the field to monitor plant disease (pathogen and pest) and stress (e.g., draught, salinity, etc.). There is a need for technology to automate environmental control in greenhouse applications. There is a need for real-time analysis of plant VOC markers and other microclimate parameters at the same time. There is a need for a multi-channel detection system that allows for the capture of plant diseases or stresses at an earlier stage. There is a need for a truly multifunctional and real-time sensor device that can track both biochemical (e.g., plant VOCs) and biophysical (e.g., temperature, humidity, etc.) signals of the plant and/or surrounding environments with high sensitivity and specificity.

There is a need to be able to simultaneously measure both chemical signals (e.g. VOC) and physical parameters (e.g., temperature and humidity). In this regard, sensor technologies for early disease diagnosis are essential to shorten stakeholder response time, identify threat before pathogen spreads, and reduce pesticide usage by optimizing application timing and choice of pesticides.

### SUMMARY

According to an embodiment of the present invention, a leaf-attachable multifunctional wearable sensor patch is provided The sensor patch comprises at least two active sensors. The at least two active sensors comprise a) at least one biochemical sensor and b) at least one biophysical sensor. The sensor patch is attachable to an abaxial leaf surface. The active sensors operate simultaneously and continuously.

According to another embodiment of the present invention, a process for making a multifunctional wearable sensor patch is provided. The sensor patch comprises at least two active sensors, and the active sensors comprise at least one biochemical sensor and at least one biophysical sensor. The at least on biochemical sensor comprises a volatile organic compound (VOC) sensor. The process comprises the steps of a) preparing the sensor patch, b) preparing the VOC sensor, c) obtaining at least one biophysical sensor, and d) assembling the sensor patch. The process for preparing the sensor patch comprises 1) obtaining silver nanowires AgNWs, 2) spray coating the AgNWs in a PMDS solution on a polyamide (PI) substrate using a stencil mask for patterning interdigitated electrodes and interconnect, and 3) removing the PI substrate when the PMDS is fully cured, to form a patterned AgNWs on PMDS substrate. The process for preparing the VOC sensor comprises 1) obtaining gold-coated silver nanowires (Au@AgNWs), 2) functionalizing the Au@AgNWs with a chemical ligand such as halothiophenol to produce a functionalized Au@AgNWs, 3) combining the functionalized Au@AgNWs with carbon nanotubes (CNTs) to form a hybrid network of the functionalized Au@AgNWs the with CNTs, and 4) obtaining a sol-gel and forming a sol-gel film over the hybrid network. The at least one biophysical sensor is selected from the group consisting of a humidity sensor, a temperature sensor, a light intensity sensor and/or a strain sensor. The process for assembling the sensor patch comprises 1) selectively depositing the at least on biochemical sensor and the at least one biophysical sensor on the patterned AgNWs on PMDS substrate, and 2) attaching at least one adhesive device to the PMDS substrate on the side of the integrated sensors, wherein the adhesive device is useful for attaching the sensor patch to a leaf.

According to yet another embodiment of the present invention, a process for monitoring plant data is provided. The process for monitoring plant data comprises a) attaching a multifunctional sensor patch comprising at least two active sensors to an abaxial leaf surface, and b) collecting the measured data by monitoring the signals from each of the active sensors. The active sensors comprise at least one biochemical sensor and at least one biophysical sensors. The active sensors operate simultaneously and continuously.

### BRIEF DESCRIPTION OF THE DRAWINGS

The present disclosure is illustrated and described herein with reference to the various drawings, in which like reference numbers are used to denote like system components/method steps, as appropriate, and in which:

FIG. 1a is a schematic of a non-limiting embodiment of the plant wearable sensor patch;

FIG. 1b is a schematic of a side-view of a non-limiting embodiment of the plant wearable sensor patch attached to a leaf;

FIG. 2a is a graph of electrical resistance changes of different surface ligand-functionalized VOC sensors (e.g., FTP, CTP, BTP, and ITP) under exposure of acetone;

FIG. 2b is a graph of electrical resistance changes of different surface ligand-functionalized VOC sensors (e.g., FTP, CTP, BTP, and ITP) under exposure of hexanal;

FIG. 2c is a graph of electrical resistance changes over time of the VOC sensor in an environment of 90% relative humidity both with and without the sol-gel layer;

FIG. 2d is a graph of electrical resistance changes over time of the VOC sensor at various temperatures with the hydrophobic sol-gel layer;

FIG. 3a is a graph of resistance changes of the temperature sensor under increasing temperature with various mixing ratio of PDMS to curing agent, the inset is an optical image of the actual temperature sensor;

FIG. 3b is a graph of electrical resistance changes versus the temperature for the temperature sensor, the temperature response was measured under relative humidity (RH) of 25%, 50%, and 75% or in the presence of 500 ppm acetone vapor (Error bar represents n=3 measurements);

FIG. 3c is a graph of capacitance changes of the humidity sensor under increasing humidity with various thickness of Nafion film, the inset is an optical image of the actual humidity sensor;

FIG. 3d is a graph of capacitance changes under increasing humidity for the humidity sensor, measured at diverse temperatures (10° C., room temperature (RT), and 40° C.) or in the presence of 500 ppm acetone vapor;

FIG. 3e is a graph comparing stomata density between the upper and lower surfaces of a leaf;

FIG. 3f is a graph of output signal differences of leaf surface humidity and VOC sensors with different sensor attachment positions;

FIG. 3g is a graph of real-time monitoring of leaf surface relative humidity and leaf surface temperature of a healthy tomato plant;

FIG. 4 is graphs of real-time sensor data of VOC emission, leaf surface humidity, environmental humidity, and leaf surface temperature from a healthy tomato plant exposed to various abiotic stresses;

FIG. 5a is graphs of real-time sensor data of VOC emission, leaf surface humidity, environmental humidity, and leaf surface temperature from a healthy tomato plant exposed to biotic stress;

FIG. 5b is a graph of real time LAMP assay results, verifying the presence of TSWV pathogens after different days of inoculation;

FIG. 5c is a graph of VOC sensor data of a healthy tomato plant exposed to Alternatria linariae (early blight) and the conventional Horsfall-Barratt scale (black line) after different days of inoculation;

FIG. 6a is a three-dimensional graph showing PCA analysis from day 0 to 15 for the 6-sensor combination (VOC_C1, C2, F1, F2, H, T), VOC_C1, C2, F1, F2, H, and T denote for 4 VOC sensors, leaf surface relative humidity sensor, and leaf temperature sensor, respectively;

FIG. 6b is a graph of the average discriminability values with different numbers of sensors as a function of infection days; and

FIG. 6c is a graph of discriminability with the best sensor composition for each number of sensors. VOC_C1, C2, F1, F2, H, and T denote for 4 different types of VOC sensors, leaf surface relative humidity sensor, and leaf temperature sensor, respectively

### DETAILED DESCRIPTION

An embodiment of the present invention provides a leaf-attachable multifunctional wearable sensor patch. The sensor patch comprises at least two active sensors. The at least two active sensors comprise a) at least one biochemical sensor and b) at least one biophysical sensor. The sensor patch is attachable to an abaxial leaf surface. The active sensors operate simultaneously and continuously.

The present invention may be understood more readily by reference to the following detailed description of the invention taken in connection with the accompanying drawing figures, which form a part of this disclosure. It is to be understood that this invention is not limited to the specific devices, methods, conditions, or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

It is to be understood that the mention of one or more method steps does not preclude the presence of additional method steps before or after the combined recited steps or intervening method steps between those steps expressly identified. Moreover, the lettering of method steps or ingredients is a conventional means for identifying discrete activities or ingredients and the recited lettering can be arranged in any sequence, unless otherwise indicated.

As used herein, the term “and/or”, when used in a list of two or more items, means that any one of the listed items can be employed by itself, or any combination of two or more of the listed items can be employed. For example, if a composition is described as containing compounds A, B, “and/or” C, the composition may contain A alone; B alone; C alone; A and B in combination; A and C in combination; B and C in combination; or A, B, and C in combination.

The sensor patch comprises at least two active sensors. At least one active sensor is a biochemical sensor and at least one active sensor is a biophysical sensor. The sensor patch is preferably attached to an abaxial leaf surface to maximize output signals from the plants. The active sensors operate simultaneously and continuously. In some aspects the active sensors comprise at least 1 or at least 2 or at least 3 or at least 4 or at least 5, or at least 6 biochemical sensors. In some aspects, the active sensors comprise at least 1 or at least 2 or at least 3 or at least 4 or at least 5, or at least 6 biophysical sensors. In some aspects, the active sensors comprise 1 to 10, 1 to 8, 1 to 6, 1 to 5, 1 to 4, 1 to 3 or 1 to 2 biochemical sensors. In some aspects, the active sensors comprise 1 to 10, 1 to 8, 1 to 6, 1 to 5, 1 to 4, 1 to 3 or 1 to 2 biophysical sensors. In some aspects, the sensor patch comprises at least 2, at least 3, at least 4, at least 5, at least 7, at least 10, at least 12 or at least 15 active sensors.

In some aspects, the at least one biochemical sensor is selected from the group consisting of a volatile organic compound (VOC) sensor, a hormone sensor, and/or a metabolite sensor. In some aspects, the biochemical sensor comprises at least 1, at least 2, at least 3, at least 4, at least 5 or at least 8 VOC sensors. In some aspects, the biochemical sensor comprises 1 to 8; or 1 to 5; or 1 to 4; or 1 to 3; or 2 to 8; or 2 to 5; or 2 to 4; or 3 to 8; or 3 to 5 VOC sensors.

In some aspects, the at least one biophysical sensor is selected from the group consisting of a humidity sensor, a temperature sensor, a light intensity sensor and/or a strain sensor. In some aspects, the humidity sensor comprises a leaf surface humidity sensor and/or an environmental humidity sensor. In some aspects, the temperature sensor comprises a leaf surface temperature sensor and/or an environmental temperature sensor.

In some aspects, the at least two active sensors are connected by flexible electrodes on a substrate. In some aspects, the flexible electrodes are selected from the group consisting of carbon nanotubes (CNT), silver nanowire (AgNW) interconnects, liquid metals, conducting polymers, graphite, and/or graphene. In some aspects, the flexible electrodes comprise AgNW. In some aspects, the substrate comprises Polydimethylsiloxane (PDMS), rubber, polyurethane, and/or styrene-ethylene-butylene-styrene (SEBS). In some aspects, the activate sensors are connected flexible by nanowire (AgNW) interconnects on a substrate comprising PDMS.

In some aspects, the at least one biochemical sensor comprises a VOC emissions sensor. In some aspects, the VOC sensor comprises a hybrid network, wherein the hybrid network comprises a first component and second component. In some aspects, the first component comprises functionalized gold-coated silver nanowires (Au@AgNWs) and/or functionalized gold nanoparticles. In some aspects, the second component comprises carbon nanotubes (CNTs), graphite, and/or graphene. In some aspects, the hybrid network comprises functionalized Au@AgNWs and CNTs.

In some aspects, the at least one biochemical sensor comprises a VOC sensor. In some aspects, the VOC sensor comprises a hybrid network, wherein the hybrid network comprises a first component and a second component. In some aspects, the first component comprises a functionalized gold-coated silver nanowires (Au@AgNWs), and/or a functionalized gold nanoparticles. In some aspects, the second component comprises a carbon nanotubes (CNTs), graphite and/or a graphene. In some aspects, the first component comprises functionalized gold-coated silver nanowires (Au@AgNWs) and the second component comprises carbon nanotubes (CNTs).

In some aspects, the functionalized Au@AgNWs, and/or the functionalized gold nanoparticles comprises a surface chemical ligand such as halothiophenol ligand, wherein the halothiophenol ligand is selected from the group consisting of fluorothiophenol (FTP) ligand, chlorothiophenol (CTP) ligand, bromothiophenol (BTP) ligand, and iodothiophenol (ITP) ligand. In some aspects the functionalized Au@AgNWs and/or the functionalized gold nanoparticle, comprise a halothiophenol ligand, wherein the halothiophenol ligand is selected from the group consisting of fluorothiophenol (FTP) ligand, chlorothiophenol (CTP) ligand. In some aspects the hybrid network comprises the functionalized Au@AgNWs, and the functionalized Au@AgNWs comprises fluorothiophenol (FTP) ligand and chlorothiophenol (CTP) ligand, bromothiophenol (BTP) ligand, and iodothiophenol (ITP) ligand. In some aspects the hybrid network comprises the functionalized Au@AgNWs and the functionalized Au@AgNWs comprises fluorothiophenol (FTP) ligand and chlorothiophenol (CTP) ligand. In some aspects, the biochemical sensor comprises the VOC sensor, and the hybrid network is covered by a hydrophobic sol-gel-layer.

When the sensor patch comprises more than one VOC sensors, the VOC sensors can comprise the same types and/or amounts of chemical ligands or different types and/or amounts of chemical ligands. In some aspects, the biochemical sensor comprises a first VOC sensor and a second VOC sensor, wherein the first VOC sensor comprises the FTP ligand and the second VOC sensor comprises the CTP ligand.

In some aspects, the at least one biophysical sensor comprises the leaf surface temperature sensor and/or the environmental temperature sensor. In some aspects, the leaf surface temperature sensor and/or the environmental temperature sensor comprises Au@AgNWs.

In some aspects, the at least one biophysical sensor comprises the leaf surface humidity sensor and/or the environmental humidity sensor, and wherein the leaf surface humidity sensor and/or the environmental humidity sensor comprises an ionomeric film. In some aspect the ionomeric film comprises Nafion. In some aspects the at least one biophysical sensor comprises the leaf surface humidity sensor and/or the environmental humidity sensor, and the leaf surface humidity sensor and/or the environmental humidity sensor comprises a film incorporating perfluorovinyl ether groups terminated with sulfonate groups onto a tetrafluoroethylene (PTFE) backbone.

According to another embodiment of the present invention, a process for making a multifunctional wearable sensor patch is provided. The sensor patch comprises at least two active sensors, and the active sensors comprise at least one biochemical sensor and at least one biophysical sensor. The at least on biochemical sensor comprises a volatile organic compound (VOC) sensor. The process comprises the steps of a) preparing the sensor patch, b) preparing the VOC sensor, c) obtaining at least one biophysical sensor, and d) assembling the sensor patch. The process for preparing the sensor patch comprises 1) obtaining silver nanowires AgNWs, 2) spray coating the AgNWs in a PMDS solution on a polyamide (PI) substrate using a stencil mask for patterning interdigitated electrodes and interconnect, and 3) removing the PI substrate when the PMDS is fully cured, to form a patterned AgNWs on PMDS substrate. The process for preparing the VOC sensor comprises 1) obtaining gold-coated silver nanowires (Au@AgNWs), 2) functionalizing the Au@AgNWs with a halothiophenol ligand to produce a functionalized Au@AgNWs, 3) combining the functionalized Au@AgNWs with carbon nanotubes (CNTs) to form a hybrid network of the functionalized Au@AgNWs the with CNTs, and 4) obtaining a sol-gel and forming a sol-gel film over the hybrid network. The at least one biophysical sensor is selected from the group consisting of a humidity sensor, a temperature sensor, a light intensity sensor and/or a strain sensor. The process for assembling the sensor patch comprises 1) selectively depositing the at least on biochemical sensor and the at least one biophysical sensor on the patterned AgNWs on PMDS substrate, and 2) attaching at least one adhesive device to the PMDS substrate on the side of the integrated sensors, wherein the adhesive device is useful for attaching the sensor patch to a leaf.

In some aspects, the functionalized Au@AgNWs comprises a halothiophenol ligand. In some aspects, the halothiophenol ligand is selected from the group consisting of fluorothiophenol (FTP) ligand, chlorothiophenol (CTP) ligand, bromothiophenol (BTP) ligand, and iodothiophenol (ITP) ligand. In some aspects the functionalized Au@AgNWs comprises a halothiophenol ligand, and the halothiophenol ligand is selected from the group consisting of fluorothiophenol (FTP) ligand and chlorothiophenol (CTP) ligand.

In some aspects, the at least one biophysical sensor comprises a leaf surface temperature sensor and/or an environmental temperature sensor. In some aspects the leaf surface temperature and/or the environmental temperature sensor comprises Au@AgNWs.

In some aspects, the at least one biophysical sensor comprises the leaf surface humidity sensor and/or the environmental humidity sensor. In some aspects, the leaf surface humidity sensor and/or the environmental humidity sensor comprises an ionomeric film. In some aspect the ionomeric film comprises Nafion. In some aspects, the at least one biophysical sensor comprises the leaf surface humidity sensor and/or the environmental humidity sensor, and the leaf surface humidity sensor and/or the environmental humidity sensor comprises a film incorporating perfluorovinyl ether groups terminated with sulfonate groups onto a tetrafluoroethylene (PTFE) backbone.

According to yet another embodiment of the present invention, a process for monitoring plant data is provided. The process for monitoring plant data comprises a) attaching a multifunctional sensor patch comprising at least two active sensors to an abaxial leaf surface, and b) collecting the measured data by monitoring the signals from each of the active sensors. The active sensors comprise at least one biochemical sensor and at least one biophysical sensors. The active sensors operate simultaneously and continuously.

In some aspects, the at least one biochemical sensor comprise volatile organic compound (VOC) sensor. In some aspects, the least one biophysical sensor is selected from the group consisting of a humidity sensor, a temperature sensor, a light intensity sensor and/or a strain sensor. In some aspects, the humidity sensor comprises a leaf surface humidity sensor and/or an environmental humidity sensor. In some aspects, temperature sensor comprises a leaf surface temperature sensor and/or an environmental temperature sensor.

In some aspects, the VOC sensor comprises a hybrid network, wherein the hybrid network comprises a first component and a second component. In some aspects, the first component comprises of a functionalized gold-coated silver nanowires (Au@AgNWs) and/or a functionalized gold nanoparticles. In some aspects, the second component comprises carbon nanotubes (CNTs), graphite and/or graphene. In some aspects the functionalized Au@AgNWs and/or the functionalized gold nanoparticles comprise a halothiophenol ligand. In some aspects, the halothiophenol ligand is selected from the group consisting of fluorothiophenol (FTP) ligand, chlorothiophenol (CTP) ligand, bromothiophenol (BTP) ligand, and/or iodothiophenol (ITP) ligand.

In some aspects, the at least one biophysical sensor comprises the leaf surface temperature sensor and/or the environmental temperature sensor. In some aspects, the leaf surface temperature and/or the environmental temperature sensor comprises Au@AgNWs.

In some aspects, the at least one biophysical sensor comprises the leaf surface humidity sensor and/or the environmental humidity sensor. In some aspects, the leaf surface humidity sensor and/or the environmental humidity sensor comprises an ionomeric film. In some aspect the ionomeric film comprises Nafion. In some aspects the at least one biophysical sensor comprises the leaf surface humidity sensor and/or the environmental humidity sensor, and the leaf surface humidity sensor and/or the environmental humidity sensor comprises a film incorporating perfluorovinyl ether groups terminated with sulfonate groups onto a tetrafluoroethylene (PTFE) backbone.

### Examples

All materials and reagents were used without further purification. Multi-walled carbon nanotubes (MWCNTs), Nafion, hexanal, and acetone were purchased from Sigma-Aldrich. Polydimethylsiloxane (PDMS, Sylgard 184) was purchased from DOW Corning.

The gold-coated silver nanowires (Au@AgNWs) were synthesized by a modified chemical solvent method. The silver nanowires (AgNWs) were prepared by a modified polyol method and dispersed in DI water for the following steps.

Preparation of solution A: 10 mL of 10 mg/mL AgNW aqueous solution, 70 mL of 5 wt % polyvinylpyrrolidone aqueous solution (PVP, Mw 40,000, Sigma Aldrich), 14 mL of 0.5 M 1-ascorbic acid aqueous solution (1-AA, Sigma Aldrich), 14 mL of 0.5 M sodium hydroxide (NaOH, Sigma Aldrich), 3.5 mL of 0.1 M Na₂SO₃ (Na₂SO₃, Sigma Aldrich) aqueous solution, and 80 mL DI water was uniformly mixed with a glass rod. The prepared solution was denoted as Solution A.

Preparation of solution B: 10 mL of 0.1 M sodium sulfite aqueous solution, 3.5 mL of 0.5 M sodium hydroxide aqueous solution, and 100 mL of DI water were mixed first. Then, 1.5 mL of 0.25 M hydrogen tetrachloroaurate (iii) hydrate aqueous solution (HAuCl₄·xH₂O, Sigma Aldrich) was added to the mixed solution. The solution was then stirred gently with a glass rod. The prepared solution is denoted as Solution B.

Solution B was then immediately but slowly poured into Solution A to produce a mixture that appeared light purple with a metallic gloss. Then, the beaker of the mixed solution was sealed and left for 12 hours. After the reaction, the Au-coated AgNWs turned light brown and precipitated out of the solution. The remaining solution turned clear black. These nanowires were then collected and washed with 95% ethanol 3 times using centrifugation (1,000 rpm for 1 minute) and dispersed in ethanol for further use.

The VOC emission sensor's performance depends heavily on the amount of ligands attached to the surface of the nanomaterials. To optimize the concentration and amount of the ligands decorated to the surface of gold-coated silver nanowires, four different concentrations (0.01 μM, 0.1 μM, 1 μM, and 10 μM) of FTP ligands were tested. We found a higher tendency of agglomeration of the nanowire solutions at the higher ligand concentration. However, if the amount of surface ligand is too low, the reactivity towards VOC analytes will also be reduced. Therefore, 0.1 μM ligand solutions were eventually chosen to functionalize gold-coated silver nanowires without significant aggregation. After that, we optimized the amount of ligand solutions by changing the volume of ligand solutions (e.g., 300, 500, and 1,000 μL). The 500 μL solution was selected to balance the ligand density and nanomaterial stability. Similarly, 500 μL of 100 nM ITP, BTP, and CTP ligand solutions were utilized to prepare other VOC sensors. Every functionalization reaction was continued for 8 hours with slight shaking at room temperature. The supernatant was then collected to measure their UV-VIS spectrum. After that, ligand-attached Au@AgNW solutions were added to MWCNTs at a 5:1 mixing weight ratio.

The sol-gel film was prepared by mixing methyltrimethoxysilane (MTMS), Tetramethylorthosilicate (TMOS), methanol, and nano-pure water in the molar ratio of 1:1:11:5. The solution was stirred at room temperature for 2 h. The final formulation was diluted ten times by adding methanol before drop-casting.

AgNWs were spray-coated on the polyimide (PI) substrate using a stencil mask for patterning interdigitated electrodes and interconnect. After patterning, PDMS solution was poured to transfer AgNWs from PI to PDMS. The PI substrate was removed when the PDMS was fully cured. With a patterned AgNW substrate, sensing materials for each sensor (e.g., Au@ AgNWs for temperature sensor, Nafion for leaf surface humidity sensor and environmental humidity sensor, and functionalized Au@AgNW+MWCNTs for VOC sensors) were selectively deposited onto the interdigitated electrodes.

The electrical resistance changes were measured by a digital multimeter (DAQ970A, Keysight) and recorded by the software Bench Vue 2018. The capacitance variation was captured by Capacitive to Digital Converter Evaluation Module (FDC1004EVM, Texas instruments). The temperature was controlled by a hotplate. Humidity was produced with a wet nitrogen gas stream. VOC gases were generated by bubbling nitrogen gas through the corresponding organic solvents. The concentration of VOC vapors was modulated by MKS mass flow controllers. For VOC sensor measurement, the sensor was exposed to VOC vapors at a fixed concentration for 2 min, followed by pure nitrogen gas purging for another 2 min for baseline recovery. Morphologies of sensing materials were measured by scanning electron microscopy (SEM, ThermoFisher Quanta 3D FEG), transmittance electron microscopy (TEM, ThermoFisher Talos F200X), and energy dispersive spectrometry (EDS, ThermoFisher Scientific SuperX EDS with the four Silicon Drift Detectors (SDD)). Sensors were attached onto the leaf surface using double-sided tape (2477p, 3M™) and connected to the data acquisition system with thin copper wires (FIXFANS) and silver adhesive epoxy (MG Chemicals) for interconnection. A commercial sensor device (Amprobe, THWD-5) was used for measuring relative humidity and temperature of the environment. A leaf porometer (Decagon Device INC, SC-1 leaf porometer) was used to measure the stomatal conductance, leaf surface humidity, and leaf surface temperature of the tomato plants under various abiotic stress conditions to validate the sensor measurement. For the reproducibility of porometer measurement, two healthy tomato plants (5-6 weeks old) for each stress condition were used, and for each plant three different leaflets were measured to minimize signal variation. In total, six measurements (from six different leaflets) were performed for each stressor and totally 12 plants were used for the experiments. For each measurement, the porometer data were collected every 30, 60, or 120 mins, and the measurements were continued for 2-4 days for different stressors.

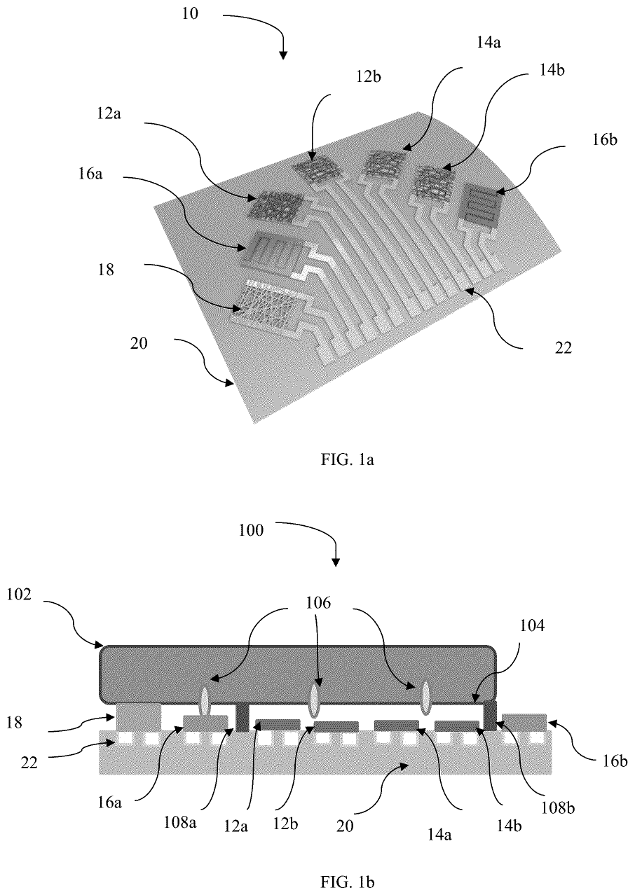

FIG. 1a is a schematic of a non-limiting embodiment of a sensor patch 10. The sensor patch 10 in FIG. 1a was used in the tomato plant examples described below. The sensor patch 10 has four VOC sensors 12a,b and 14a,b comprising Au@agNW-ligands, MWCNT, and a sol-gel layer. The sensor patch 10 has two VOC sensor patches with chlorothiophenol (CTP) ligand 12a and 12b (labeled VOC_C1 and VOC_C2), and two VOC sensor patches with fluorothiophenol (FTP) ligand 14a and 14b (labeled VOC_F1 and VOC_F2). The Nafion leaf surface humidity sensor 16a is next the VOC_C1 sensor 12a and the Nafion ambient humidity sensor 16b is next to the VOC_F2 sensor 14b. The leaf surface temperature sensor 18 comprises Au@AgNW and is located next to leaf surface humidity sensor 16a. The sensors 12a,b, 14a,b, 16a,b, and 18 are located on a substrate 20 which is made of PMDS and interconnected by a network 22 of electrodes and AgNWs.

FIG. 1b is a schematic 100 of the sensor patch 10 attached to a leaf 102. Specifically, sensor patch 10 is attached to the leaf underside 104 where the concentration of stomata 106 is highest. The leaf surface temperature sensor 18 is shown abutting the leaf underside 104 with spacer/adhesive objects 108a,b attaching the sensor patch 10 to the leaf underside 104 while maintaining a small space between the sensor patch 10 and the leaf surface humidity sensor 16a and the four VOC sensors 12a,b and 14a,b. The sensor patch 10 is attached to the leaf underside 104 such that the environmental humidity sensor 16b is not covered by the leaf 102.

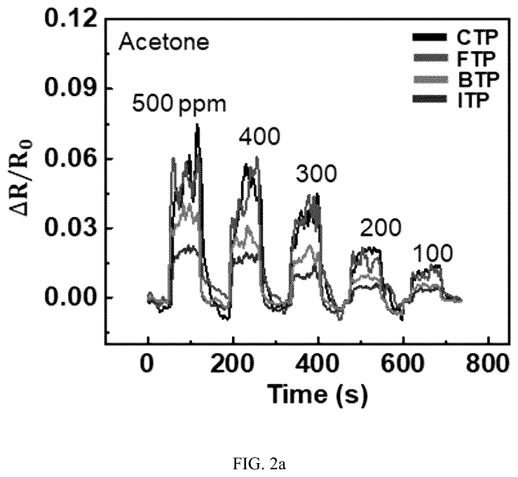

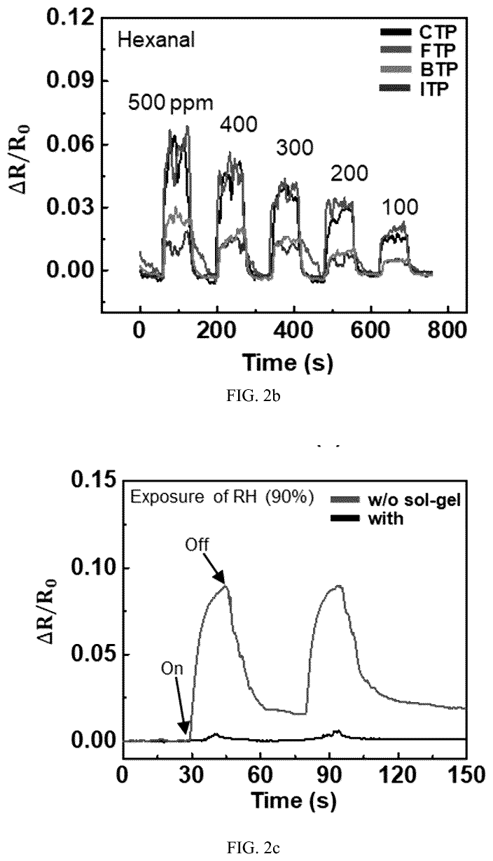

We tested the performance of the VOC sensors (namely FTP, CTP, BTP, and ITP sensors) by measuring the electrical resistance under the exposure of acetone or hexanal vapors, which are used to mimic ketone and aldehyde-based VOCs emitted from the plant (FIG. 2a, 2b). The VOC sensors are operated as chemiresistive sensors, where the electrical resistance of MWCNTs varies upon the attachment of VOC molecules on its surface, which creates a doping effect on the carbon nanomaterials. When solvent vapor concentration was reduced from 500 ppm to 100 ppm, the sensor response (ΔR/R₀) decreased accordingly, as a result of the reduced interaction between VOC gas and sensing materials (FIG. 2a, b). Furthermore, for both acetone and hexanal vapors, the sensitivity of FTP and CTP sensors is the highest among the four, followed by BTP and ITP sensors. This is due to the decreasing electronegativity of the halogen atoms, following the order of F>C>B>I. For the final wearable sensor patch, only FTP and CTP VOC sensors (termed VOC_F, and VOC_C, respectively) were integrated due to their higher detection sensitivity.

To characterize the potential crosstalk from other stimuli such as humidity and temperature, the VOC sensors were tested under different humidity and temperature conditions. As shown in FIG. 2c, the presence of a hydrophobic and gas-permeable sol-gel layer on the top of the VOC sensors can prevent the interference of water molecules up to 90% relative humidity (RH) (FIG. 2c, bottom curve), whereas the control sensor without the sol-gel coating showed a significant response to the environmental humidity (FIG. 2c, top curve). The sol-gel layer also improved the repeatability of the VOC sensor response by enhancing the stability of the sensing materials on PDMS substrate and reducing baseline signal drifting. For temperature interference, the VOC sensor showed minimal dependence on the external temperature (FIG. 2d), owing to the opposite temperature coefficient of resistance (TCR) values of MWCNTs (−0.33%/K) and AgNWs (0.26%/K). Although the resistance of VOC sensors slightly fluctuated with the temperature variation (from 40 to 60° C.), this change was less than 0.7% for 40° C. and lower temperatures (FIG. 2d), exhibiting acceptable temperature stability for real plant applications.

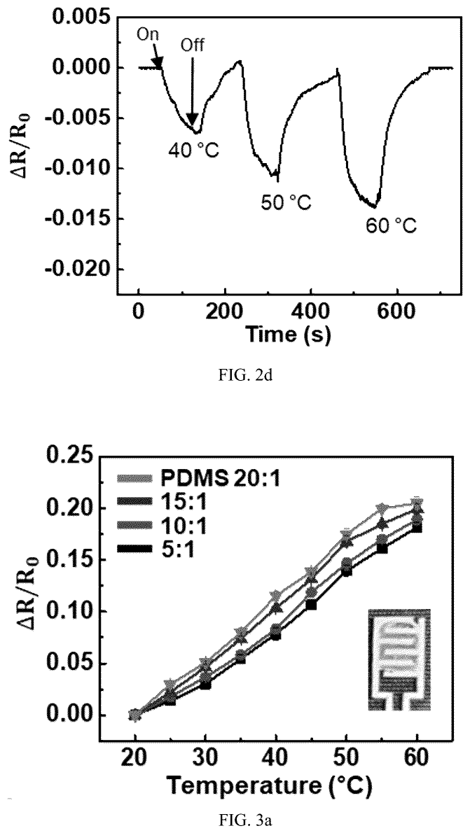

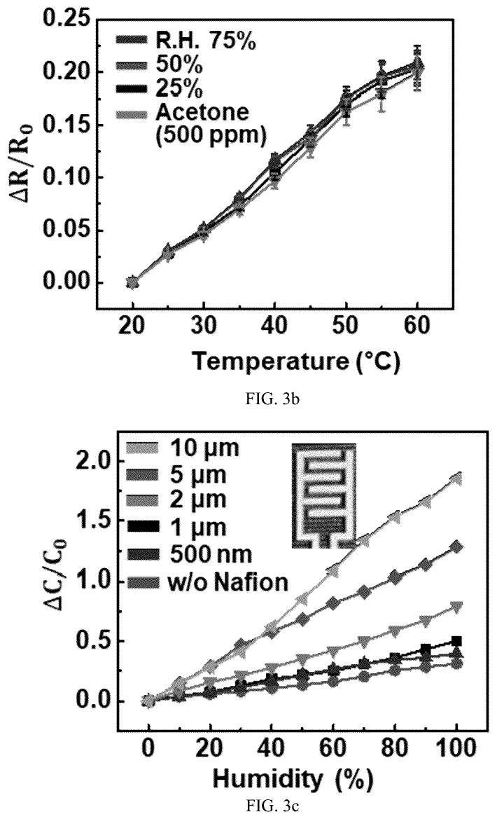

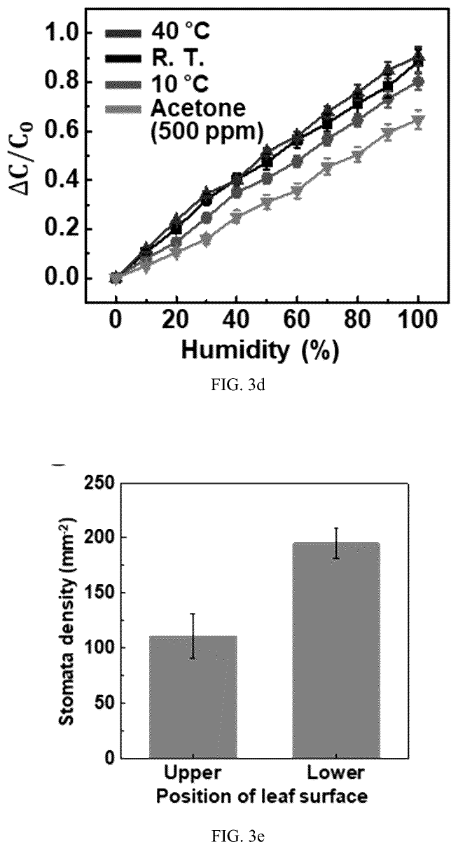

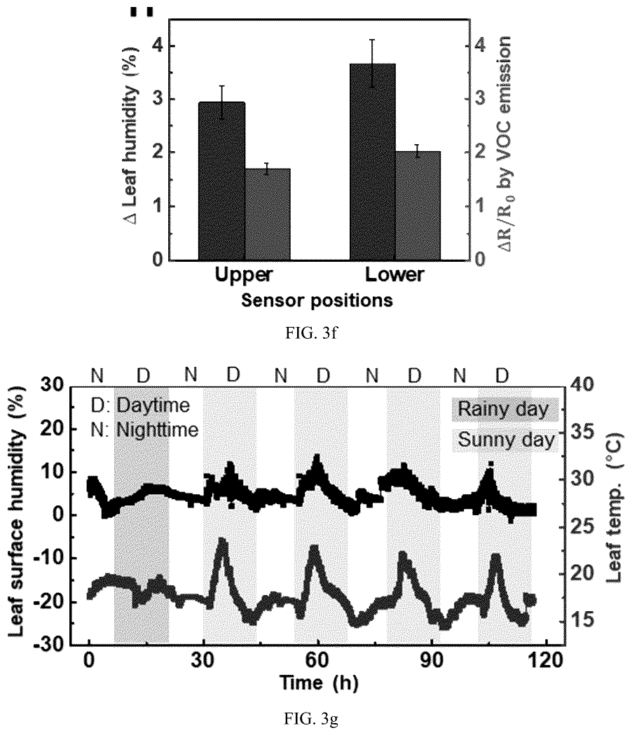

FIG. 3a shows the sensing performance of the leaf temperature sensor, composed of as-synthesized Au@AgNWs (without functionalization) as the sensing agent. Due to the temperature sensitivity of Au@AgNWs and the thermal expansion of PDMS substrate, the nanowire-based temperature sensor can detect temperature changes from room temperature to 60° C. To investigate the effect of PDMS thermal expansion on temperature sensing, we varied the elastic modulus of PDMS by tuning the mixing ratio of prepolymer and curing agent from 5:1 to 20:1 (FIG. 3a). As the mixing ratio increased to 20:1 (resulting in softer PDMS), the sensitivity of the temperature sensor slightly increased due to larger thermal expansion of PDMS, which decreased the percolation between Au@AgNWs. Moreover, the interference of different humidity levels or solvent exposure to the temperature sensor was also examined (FIG. 3b). The slope of each response curve did not change significantly when various humidity levels (RH 25%, 50%, and 75%) or 500 ppm of acetone were applied, indicating the excellent stability of the temperature sensor. These results can be explained by the protective effect of the gold layer on the AgNW core, which does not interact with water or common VOC vapors due to its chemical inertness.

The performance of the humidity sensors was also characterized. In this case, Nafion film was utilized as the active humidity sensing material by measuring its capacitance changes between two electrodes. The thickness of the Nafion film was controlled to optimize the detection sensitivity (FIG. 3c). Nafion is known to absorb water molecules, resulting in an increase in the capacitance value by replacing air with water molecules within the film. As the thickness of the film was increased, the sensitivity was enhanced due to the higher capacity of absorbing water molecules (FIG. 3c). However, when the thickness was above 5 μm, the film was delaminated from the substrate due to a larger stiffness mismatch between the film and the substrate. Therefore, a 2-μm thick Nafion layer was selected as the optimized thickness. To test the cross interference, we applied various temperatures (10° C., room temperature, and 40° C.) or 500 ppm of acetone vapor to the humidity sensor (FIG. 3d). With the increase of temperature, larger thermal movement of water molecules would induce higher polarization, resulting in a larger capacitance. This means that the absolute capacitance value (C) would change as a function of the temperature. However, the relative capacitance response (□C/C₀) is quite consistent for temperatures between 10° C. and 40° C. In the case of acetone exposure, the sensitivity of the humidity sensor was reduced slightly due to water molecule absorption in the presence of acetone.

Next, the influence of the sensor location, namely the adaxial (upper surface of leaf) attachment versus the abaxial (lower surface of leaf) attachment, was evaluated on a live tomato plant. As shown in FIG. 3e, the density of stomata on the abaxial surface is approximately 73% higher than that of the adaxial leaf. This result agrees with the previous study, where a higher density of stomata on the lower surface of tomato leaf was reported. For leaf surface humidity and VOC sensors, the sensor responses with abaxial surface position were on average 10˜20% higher than those of adaxial surface (FIG. 3f). Here, the leaf surface humidity sensor measured the leaf-emitted water under normal growth conditions, while the VOC sensor signal was induced by the leafy VOC profile change upon mechanical leaf cutting. The signal difference was attributed to the difference in stomata density between the upper and lower surfaces of the leaf (FIG. 3e). For better sensor performance, we therefore chose the abaxial epidermis of the leaf as our sensor attachment location for the rest of experiments in this work. In addition, we tested the wearability and biocompatibility of sensor for nearly 3 weeks on a live tomato plant. The data showed no apparent indication of plant stress after sensor attachment.

Before testing the plant stress conditions in the next section, we first monitored the leaf surface temperature and relative humidity of a healthy tomato plant during sunny and rainy days continuously using the wearable sensor patch (FIG. 3g). As the light intensity increased during the daytime, the leaf surface humidity signals reached the maximum in the middle of the day due to the increased number of open stomata to release the water molecules to the environment (FIG. 3g). This indicates that the intensity of the sunlight can modulate the opening of stomata on leaf surface. The leaf temperature increased under the sunlight condition during the daytime, followed by a decrease in temperature that is attributed to the enhanced leaf transpiration (FIG. 3g). However, such a circadian rhythm was much less obvious during rainy days when the plant was less influenced by the sunlight with high relative humidity of the air (FIG. 3g). These results show that this multimodal sensor patch can be potentially used for the study of plant chronobiology, in addition to disease and stress detection as described below.

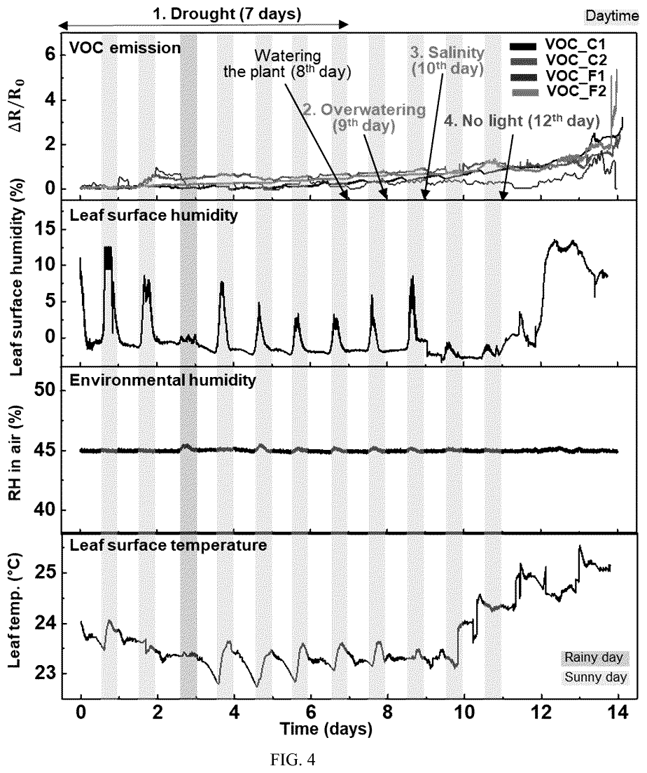

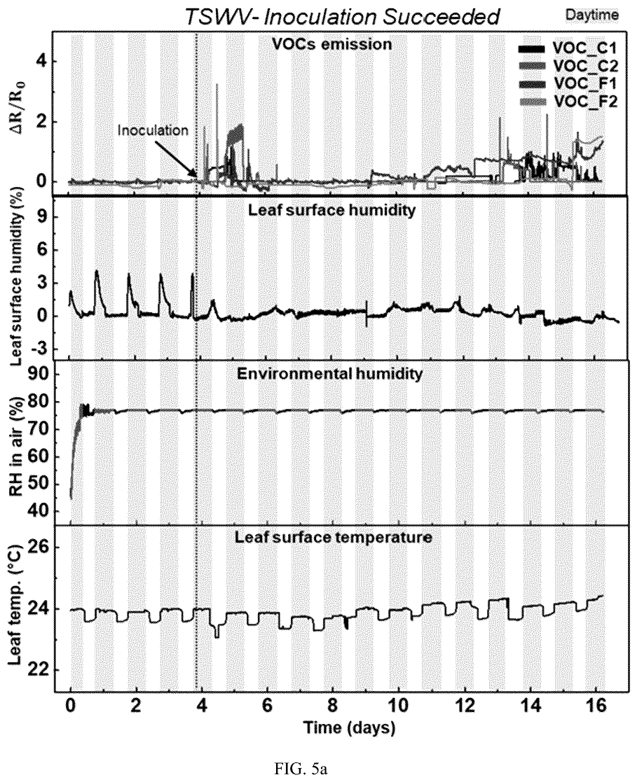

To demonstrate the usability of the multimodal sensor, the patch was mounted on a live tomato plant for monitoring various abiotic stresses, including drought, overwatering, salinity, and darkness. Four stressors were introduced sequentially to the same tomato plant wearing the sensor patch to minimize host difference. For this experiment, we used a multimodal wearable sensor composed of 4 VOC sensors (CTP sensors: VOC_C1 and C2; FTP sensors: VOC_F1 and F2), 1 leaf surface humidity sensor, 1 environmental humidity sensor, and 1 leaf surface temperature sensor (FIG. 1). By applying the sequential stimuli, the responses of the sensor with multiple electrical signals were simultaneously monitored by a multichannel data recorder for up to 14 days (FIG. 4). First, watering was prevented for 7 days to mimic a drought environment. In this stage, the leaf surface humidity gradually reduced as water was conserved inside the plant over time, while the VOC signals increased slightly (FIG. 4, 1^{st} and 2^{nd} panel). Although the plant was grown indoors, we were able to capture a rainy day (day 3) by observing the depression of leaf transpiration which was modulated by the different lightening conditions, similar to the results in FIG. 3g. After 7 days, the plant was watered normally to reduce the water stress and the plant recovered for another day before next experiments. On day 9, excessive water was applied to create an overwatering condition. Immediately, the surface humidity of leaf increased about 1.5 times as water content inside the host decreased as increased water evaporation occurred through leaf surface (FIG. 4, 2nd panel). On day 10, salinity stress was introduced by using 150 mM salted water. Typically, under high-salt conditions, the transpiration of the plant would be reduced due to the reduction of water absorption from roots as a result of the osmotic pressure difference between soil and root. This was confirmed from our experimental data, where the suppression of leaf surface humidity change and rapid increase of leaf temperature due to reduced transpiration was observed (FIG. 4, 2^{nd} and 4^{th} panel). For the last experiment (day 12 and after), the plant was placed in complete darkness by covering the plant with a box. In this case, significantly increased VOC emission, leaf surface humidity, and leaf temperature were observed, which probably resulted from the significant stress due to the lack of photosynthesis. The plant eventually died at the end of the experiment, while showing high levels of VOC emissions and increased leaf temperature. The elevated leaf surface humidity after light blocking appears contradictory to the conventional plant physiology theory. However, we attribute this phenomenon to the plausible evapotranspiration effect of a dying plant.

Susceptible tomato plants (cv. Mountain Fresh Plus) were grown from seed at the NCSU Phytotron with a combination of natural light and artificial light (14 hr photoperiod) and a 26° C. day and 22° C. night temperature cycle. Approximately one week before the experiment, five to six-week-old plants were transferred to a growth chamber with a 16-hour photoperiod kept at 23° C. (both day and night) for the entire experimental period.

One plant was selected for each inoculation. The plant was then placed in an inverted inoculation box lined with moist paper towels to provide water and humidity throughout the experiment.

An isolate of A. linariae (JD1B) was maintained on potato dextrose agar throughout the experiment. To generate conidia, pieces of agar with active A. linariae culture were broken up in potato dextrose broth and spread onto sporulation agar (0.2 g CaCO₃, 100 ml V8 juice, 20 g Difco Bacto agar, 1 L dH2O). Plates were incubated at 20° C. under constant light for two weeks. Conidia production was then stimulated by brushing the plates with a dry sterile cell spreader and incubating at room temperature for 1-2 days with the lids ajar in an inoculation box. To harvest conidia, 2 mL of sterile distilled water was added to the plate and brushed with a cell spreader. The liquid was then removed and quantified using a hemocytometer. The conidia solution was diluted to 2,000 conidia/mL using distilled water. Four plants were sprayed with 2 mL of the conidia solution over the entire surface of the plant, while four control plants were sprayed with 2 mL of distilled water. Plants were covered with clear plastic bags to maintain humidity.

In addition to monitoring by the sensor, visual symptoms were observed daily for one week by measuring the percent leaf area diseased (% LAD) using a modified Horsfall-Barratt scale.

A week prior to the experiment, a two-week-old tomato Mountain Fresh seedling, susceptible to TSWV, was placed in a growth chamber at 23° C. with a 16-h light/8-h dark schedule. The plant in a pot was also enclosed in a plastic container with paper towels soaked in a nutrient solution. As the experiment started, the seedling was inoculated with a wild-type TSWV strain that originated from California using a mechanical leaf-rub method. First, all leaves were sprinkled with carborundum. Second, several young leaves from TSWV-infected tomatoes were ground in an ice-cold mortar with approximately 5-10 mL of sodium sulfite solution (63 mg per 50 ml tap water) as a buffer. Next, two cotton applicators were repeatedly dipped into the ground tissue mix and rubbed on each leaf, using a gloved hand to support the leaf and ensure small wounds were made on the leaf surface. The leaf area with the sensor attached was avoided. Ten minutes after this procedure, the tomato plant was sprayed with DI water to remove remaining carborundum. The plant was kept enclosed in a controlled chamber for 14 days. Plants were fertilized with nutrient solution three times per week. For the negative control, mock inoculation was performed with healthy leaf tissue, and the experiment schedule was kept the same.

At the experiment termination, one young leaf was used for DNA/RNA extraction. Polymeric microneedle patch was pressed on a leaf and rinsed with 60 μL of deionized water. To detect TSWV, 25 μL LAMP reactions with EvaGreen fluorescent and HNB (Hydroxy naphthol blue) colorimetric dye were performed on a BIO-RAD CFX96 real-time machine. For each reaction, 2 μL of microneedle extraction was used for analysis. For positive controls, we used 2 μL RNA extracted with Total RNA (Plant) Kit (IBI Scientific) from symptomatic tomato plants maintained in the lab. For No Template Controls, 2 L of molecular grade DI water was added to the reactions. Positive reactions were detected by green fluorescence with Ct values recorded, and also by a color change from violet to light blue.

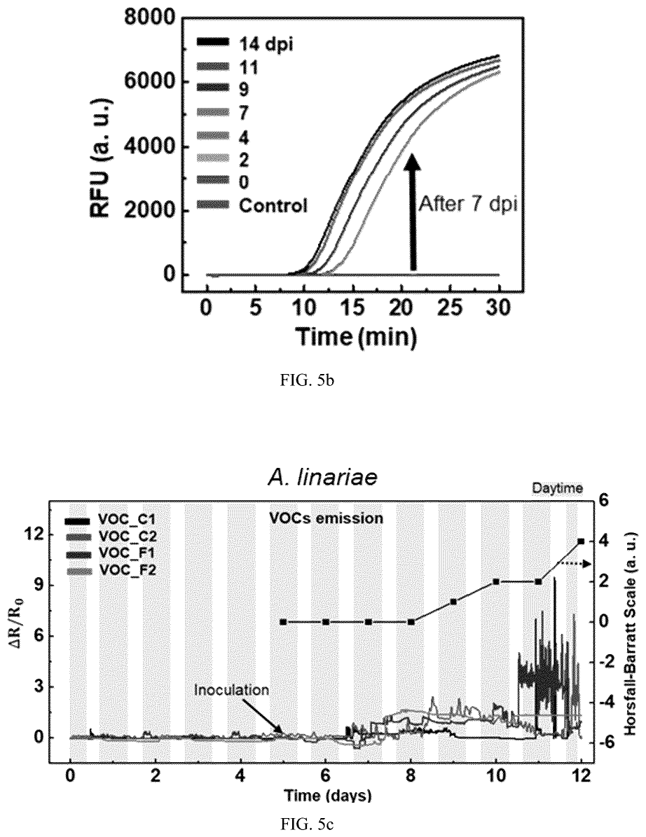

Tomato spotted wilt virus (TSWV, a viral pathogen) and early blight (Alternaria linariae, a fungal pathogen) were selected due to their prevalence in tomato production. The experiments were conducted in a growth chamber at the NCSU Phytotron, maintained at a constant 23° C. with a 16-hour photoperiod and constant carbon dioxide concentration (400 ppm). TSWV inoculation was first performed, and the plant response was monitored by the wearable sensor. By measuring the electrical signals with the wearable sensor, differentiable VOC signals can be seen after around five days post inoculation (dpi) (inoculated on day 4, and detectable VOC signals on day 9) (FIG. 5a). To compare our electrical sensing method with the conventional molecular diagnostic method, a TSWV-specific real time RT-LAMP assay was performed in parallel (FIG. 5b). According to the real time RT-LAMP assay results, successful inoculation of the plant generated consistent positive nucleic acid test results seven dpi (FIG. 5b), which is later than the wearable sensor patch. More quantitative early detection data was determined by the machine learning analysis described below. Also, a rapid change of VOC signals and a decrease in leaf surface relative humidity was captured right after the inoculation (day 4 and 5). These signal perturbations were attributed to the mechanical damage to the leaf surface during the TSWV rub-inoculation process. The VOC signals returned to the baseline on day 7, indicating the recovery of the plant from mechanical damage. In addition to VOCs, leaf surface relative humidity rapidly decreased and the leaf temperature slightly increased after inoculation (FIGS. 5a, 2nd and 4th panel), which can be related to the mechanical damage of the leaf surface and also the closure of the stomata due after pathogen infection. When a plant is infected by TSWV, stomata generally close as a result of invasion by the pathogen, resulting in a lower transpiration rate. Although less specific, these biophysical signals (e.g., leaf surface relative humidity and temperature) are easy to monitor compared to VOC signals, and hence could be useful plant health indicators on their own.

To demonstrate the feasibility of detecting fungal pathogen infection using the same sensor patch, we also performed inoculation experiments with A. linariae (FIG. 5c). Since the inoculation of this fungal pathogen was performed by spraying the pathogen solution on the leaf surface without mechanical damage, no instant VOC signal changes were observed after inoculation in this case (FIG. 5c). For A. linariae, our wearable sensor demonstrated the capability of detecting pathogen infection before the visual assessment method. Conventionally, the visual symptoms were quantified using the Horsfall-Barrratt scale for assessing disease severity. Based on the scale, the infection could be confirmed visually 4 dpi for A. linariae (FIG. 5c, black squares). However, the wearable VOC sensor was able to capture elevated VOC emissions after two days of infection, approximately two days before the visible assessment method. Leaf surface humidity and temperature signals also showed different patterns before and after infection. Together, these results (FIG. 5a,c) suggested that the wearable sensor technology was able to detect different kinds of plant pathogens (viral and fungal) 2-3 days earlier than conventional detection methods, such as the nucleic acid testing by LAMP assays FIG. 5b and the visible inspection in FIG. 5c. Our previous work also demonstrated that a wearable sensor could detect infection by the Oomycete pathogen Phytophthora infestans. As a comparison, healthy tomatoes sprayed with water instead of the spore solution showed no sensor response at all.

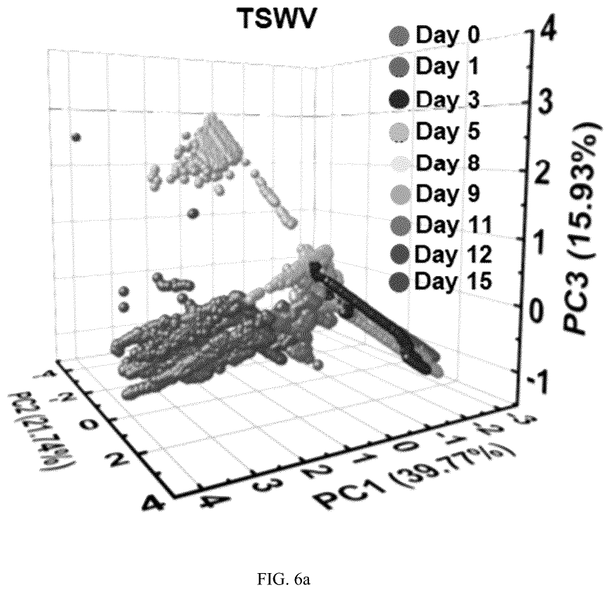

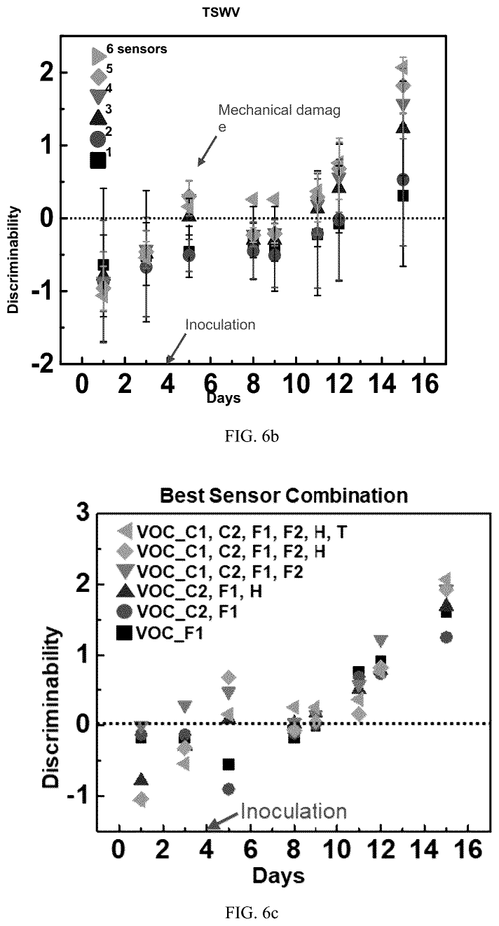

To quantitatively assess our multimodal sensors for the early detection of pathogens, an unsupervised machine learning approach based on Principle Component Analysis (PCA) was utilized to analyze the real-time sensor data (FIG. 6). PCA is one of the most well-known statistical algorithms in data analysis and image processing projects for multivariate variable dimensionality reduction with impactful benefits such as feature selection and event classification. In comparison with other conventional methods such as t-SNE approach, the PCA approach is more applicable in multivariable sensor systems. For the demonstration, we used the TSWV inoculation data (FIG. 5a) as an example. The multi-channel wearable sensor data from the same plant was first divided into different days (e.g., day 0, 1, 2, 3, etc.). Day 0 data was used as the healthy control, and compared to other days. Data from different days were clustered by PCA with reduced data dimensions. Then, the centroid and Euclidean distance between two centroids of clusters (two different days) were calculated. The separation of the clusters was quantitatively assessed by a parameter called “Discriminability” (D), as defined by the following equation.

$$D = E - ( {R}_{STD , 1} + {R}_{STD , 2} )$$

where D, E, and R denote discriminability, Euclidean distance, and radius (standard deviation) of the cluster, respectively.

As shown in FIG. 6a, a 3-component PCA analysis for total 6 sensors (namely VOC_C1, VOC_C2, VOC_F1, VOC_F2, leaf surface relative humidity sensor (H), and temperature sensor (T)) from day 0 to day 15 shows the gradual separation of sensor signals from day 0 (green dots) throughout the TSWV infection process. In the early days, the most obvious cluster separation occurred on day 5 (cyan dots) due to the mechanical damage induced by the inoculating method (FIG. 6a).

Using Discriminability (capturing different signal changes), we are able to quantitatively differentiate diseased plants from healthy controls and determine the accurate early detection day. Simply, if the Discriminability value is positive, the two sensor clusters are considered distinguishable, resulting in a positive diagnosis result. On the other side, if the value is negative, that means the two clusters under comparison are overlapped, resulting in a negative detection result. We applied the PCA analysis to all possible sensor combinations using 6 individual sensors (total 63 combinations), and calculated Discriminability for each sensor combination (FIG. 6b). FIG. 6b shows the averaged Discriminability value for each sensor combination, and the standard deviation represents the different performance when the number of sensors is fixed but the sensor composition is different. Based on the data, it clearly suggests that the more sensor channels that were used, the higher Discriminability value, which also means higher confidence in a positive detection. Excluding the day 5 data (positive but mainly due to mechanical perturbation), the 6-sensor channel combination can clearly detect TSWV infection as early as day 8, which is 4 dpi, much earlier than the RT-LAMP results (7 dpi, FIG. 5b). FIG. 6b displayed a large error bar for each fixed number of sensors, suggesting the actual sensor channel combination is equally if not more important to the total number of sensors. The best combination for each number of sensors is presented in FIG. 6c. According to the Discriminability values, minimum 3 sensors (VOC_C2, VOC_F1, and H) are needed for the early detection of TSWV after 4 dpi (FIG. 6c, blue triangle). Also, the results suggest that for effective disease detection, the biochemical VOC sensor is probably the most important sensor that is needed in each sensor combination; in addition, the leaf surface humidity sensor works slightly more effectively than the leaf temperature sensor in disease detection (FIG. 6c). Such a machine learning analysis can help find the most impactful sensor (and sensor combination) for a particular application, and potentially reduce the total number of redundant sensors, which would be particularly useful to reduce sensor cost while maintaining sensor performance.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention and are intended to be covered by the following claims.

What is claimed is:

1. A leaf-attachable multifunctional wearable sensor patch, the sensor patch comprising at least two active sensors,
wherein the active sensors comprise:
a) at least one biochemical sensor; and
b) at least one biophysical sensor,
wherein the sensor patch is attachable to an abaxial leaf surface, and
wherein the active sensors operate simultaneously and continuously.

2. The sensor patch of claim 1, wherein the at least one biochemical sensor is selected from the group consisting of a volatile organic compound (VOC) sensor, a hormone sensor, and/or a metabolite sensor,
wherein the least one biophysical sensor is selected from the group consisting of a humidity sensor, a temperature sensor, a light intensity sensor and/or a strain sensor,
wherein the humidity sensor comprises a leaf surface humidity sensor and/or an environmental humidity sensor, and
wherein the temperature sensor comprises a leaf surface temperature sensor and/or an environmental temperature sensor.

3. The sensor patch of claim 1, wherein the at least two activate sensors are connected by flexible electrodes selected from the group consisting of carbon nanotubes (CNT), nanowire (AgNW) interconnects, liquid metals, conducting polymers, graphite, and/or graphene on a substrate comprising Polydimethylsiloxane (PDMS), rubber, polyurethane, and/or styrene-ethylene-butylene-styrene (SEBS).

4. The sensor patch of claim 1, wherein the at least one biochemical sensor comprises a VOC emissions sensor, and the VOC sensor comprises a hybrid network, wherein the hybrid network comprises a first component and a second component, wherein the first component comprises a functionalized gold-coated silver nanowires (Au@AgNWs), and/or a functionalized gold nanoparticles, and wherein the second component comprises a carbon nanotubes (CNTs), graphite, and/or a graphene.

5. The sensor patch of claim 4, wherein the functionalized Au@AgNWs, and/or the functionalized gold nanoparticles, comprise a chemical ligand such as halothiophenol ligand, wherein the halothiophenol ligand is selected from the group consisting of fluorothiophenol (FTP) ligand, chlorothiophenol (CTP) ligand, bromothiophenol (BTP) ligand, and iodothiophenol (ITP) ligand.

6. The sensor patch of claim 5, wherein the biochemical sensor comprises a first VOC sensor and a second VOC sensor, wherein the first VOC sensor comprises the FTP ligand and the second VOC sensor comprises the CTP ligand.

7. The sensor patch of claim 4, wherein, the biochemical sensor comprises the VOC sensor, and wherein the hybrid network is covered by a hydrophobic sol-gel-layer.

8. The sensor patch of claim 2, wherein the at least one biophysical sensor comprises the leaf surface temperature sensor and/or the environmental temperature sensor, and wherein the leaf surface temperature sensor and/or the environmental temperature sensor comprises Au@AgNWs.

9. The sensor patch of claim 2, wherein the at least one biophysical sensor comprises the leaf surface humidity sensor and/or the environmental humidity sensor, and wherein the leaf surface humidity sensor and/or the environmental humidity sensor comprises an ionomeric film.

10. The sensor patch of claim 2, wherein the sensor patch comprises 1 to 6 biochemical sensors and 1 to 6 biophysical sensors.

11. A process for making a leaf-attachable multifunctional wearable sensor patch comprising at least two active sensors wherein the active sensors comprise at least one biochemical sensor and at least one biophysical sensor, wherein the biochemical sensor comprises a volatile organic compound (VOC) sensor, the process comprising:
a) preparing the sensor patch, the process for preparing the sensor patch comprising:
1) obtaining silver nanowires AgNWs;
2) spray coating the AgNWs in a PMDS solution on a polyamide (PI) substrate using a stencil mask for patterning interdigitated electrodes and interconnect; and
3) removing the PI substrate when the PMDS is fully cured, to form a patterned AgNWs on PMDS substrate;
b) preparing the VOC sensor, the process for preparing the VOC sensor comprising:
1) obtaining gold-coated silver nanowires (Au@AgNWs);
2) functionalizing the Au@AgNWs with a halothiophenol ligand to produce a functionalized Au@AgNWs;
3) combining the functionalized Au@AgNWs and carbon nanotubes (CNTs) to form a hybrid network of the functionalized Au@AgNWs and the CNTs; and
4) obtaining a sol-gel and forming a sol-gel film over the hybrid network;
c) obtaining at least one biophysical sensor selected from the group consisting of a humidity sensor, a temperature sensor, a light intensity sensor and/or a strain sensor; and
d) assembling the sensor patch, the process for assembling the sensor patch comprising:
1) selectively depositing the at least on biochemical sensor and the at least one biophysical sensor on the patterned AgNWs on PMDS substrate; and
2) attaching at least one adhesive device to the PMDS substrate on a side of the PMDS substrate with the active sensors, wherein the adhesive device is useful for attaching the sensor patch to a leaf.

12. The process of claim 11, wherein the functionalized Au@AgNWs comprises a halothiophenol ligand, wherein the halothiophenol ligand is selected from the group consisting of fluorothiophenol (FTP) ligand, chlorothiophenol (CTP) ligand, bromothiophenol (BTP) ligand, and iodothiophenol (ITP) ligand.

13. The process of claim 11, wherein the at least one biophysical sensor comprises a leaf surface temperature sensor and/or an environmental temperature sensor, and wherein the leaf surface temperature and/or the environmental temperature sensor comprises Au@AgNWs.

14. The process of claim 11, wherein the at least one biophysical sensor comprises the leaf surface humidity sensor and/or the environmental humidity sensor, and wherein the leaf surface humidity sensor and/or the environmental humidity sensor comprises an ionomeric film.

15. A process for monitoring plant data, the process comprising:
a) attaching a multifunctional sensor patch comprising at least two active sensors to an abaxial leaf surface, and
b) collecting the measured data by monitoring the signals from each of the active sensors;
wherein the active sensors comprise at least one biochemical sensor and at least one biophysical sensors, and
wherein the active sensors operate simultaneously and continuously.

16. The process of claim 15, wherein the at least one biochemical sensor comprises a volatile organic compound (VOC) sensor,
wherein the least one biophysical sensor is selected from the group consisting of a humidity sensor, a temperature sensor, a light intensity sensor and/or a strain sensor,
wherein the humidity sensor comprises a leaf surface humidity sensor and/or an environmental humidity sensor, and
wherein the temperature sensor comprises a leaf surface temperature sensor and/or an environmental temperature sensor.

17. The process of claim 16, wherein the VOC sensor comprises a hybrid network,
wherein the hybrid network comprises a first component and a second component,
wherein the first component comprises of a functionalized gold-coated silver nanowires (Au@AgNWs) and/or a functionalized gold nanoparticle,
wherein the second component comprises carbon nanotubes (CNTs), graphite and/or graphene, and
wherein the functionalized Au@AgNWs and/or the functionalized gold nanoparticles comprise a halothiophenol ligand,
wherein the halothiophenol ligand is selected from the group consisting of fluorothiophenol (FTP) ligand, chlorothiophenol (CTP) ligand, bromothiophenol (BTP) ligand, and/or iodothiophenol (ITP) ligand.

18. The process of claim 15, wherein the at least one biophysical sensor comprises the leaf surface temperature sensor and/or the environmental temperature sensor, and wherein the leaf surface temperature and/or the environmental temperature sensor comprises Au@AgNWs.

19. The process of claim 15, wherein the at least one biophysical sensor comprises the leaf surface humidity sensor and/or the environmental humidity sensor, and
wherein the leaf surface humidity sensor and/or the environmental humidity sensor comprises an ionomeric film.

20. The process of claim 15, wherein the sensor patch comprises 1 to 6 biochemical sensors and 1 to 6 biophysical sensors.
